(19) United States
(12) Patent Application Publication
(10) Pub. No.: US 2006/0178731 A1
(43) Pub. Date: Aug. 10, 2006

(54) Apparatus for aiding the flow of blood through patient's circulatory system

(75) Inventor: Allen J. Tower, North Lawrence, NY (US)

Correspondence Address: WALL MARJAMA & BILINSKI, 101 SOUTH SALINA STREET, SUITE 400, SYRACUSE, NY, 13202, US

(73) Assignee: Numed, Inc., Nicholville, NY (US)

(21) Appl. No.: 11/054,688

(22) Filed: Feb. 9, 2005

(51) Int. Cl. A61F 2/06

(52) U.S. Cl. 623/1.24

(57) ABSTRACT

A percutaneous implanted device for temporarily sustaining a patient awaiting a heart transplant. The device includes a radially expandable stent mounted upon the balloon section of a balloon catheter. A venous biological valve is mounted at each end of the stent and an inflatable annular-shaped diaphragm is secured to the stent between the valves, which permit the flow of blood in one direction and prevent the flow of blood in the opposite direction. The diaphragm is attached to a pump which inflates the diaphragm after the stent is implanted in a blood vessel in relation to the heart's rhythm to assist the heart in moving blood through the patient's circulatory system.

### FIELD OF THE INVENTION

This invention relates generally to a percutaneous implanted device for assisting the heart of a patient who is awaiting a heart transplant in circulating blood.

### BACKGROUND OF THE INVENTION

Recently, the Food and Drug Administration has approved the use of a temporary artificial heart for use in high risk patients who are in danger of dying within a relatively short period of time while awaiting a heart transplant. The device is designed to take over the function of the patient's failing heart to restore normal or close to normal blood pressure, and thus help the functioning of other vital organs such as the kidneys and the liver. The device must be surgically implanted within a patient who typically is weakened because of a failing heart. Accordingly, the device is suggested for use in extreme cases where patients have no other chance of survival.

Attempts have also been made in which a balloon catheter is implanted percutaneously into a patient's blood vessel leading towards or away from the heart. The balloon is pulsed in synchronization with the heart's rhythm to help push blood through the blood vessel and thus aiding the heart in circulating blood through the patient's body. Although the pulsed balloon catheter approach does help the heart in moving the flow of blood through the patient's body and avoids many of the risks associated with surgery, the efficiency of the system is relatively low.

### SUMMARY OF THE INVENTION

It is a primary object of the present invention to temporarily assist a weakened heart in passing blood through a patient's circulatory system.

It is a further object of the present invention to percutaneously implant a device for temporarily assisting the heart in circulating blood.

A still further object of the present invention is to better sustain a patient who is awaiting a heart transplant.

Another object of the p resent invention is to avoid the need of surgically implanting a temporary mechanical device for aiding a patient's weakened heart.

These and other objects of the present invention are attained by a percutaneous implanted apparatus for temporarily sustaining a patient awaiting a heart transplant. The device includes a radially expanded stent having a collapsible biological valve secured to each end of the stent so that blood can only flow through the stent in one direction. An inflatable diaphragm is secured to the stent between the valves and is attached to an exterior pump that is arranged to inflate and deflate the diaphragm. A controller monitors the heart rate of the patient and regulates the activity of the pump in relation to the diastolic and systolic rhythm of the heart. The stent in a collapsed condition is percutaneously implanted into a desired blood vessel leading to or away from the patient's heart using a balloon catheter.

### BRIEF DESCRIPTION OF THE DRAWINGS

For a better understanding of these and other objects of the invention, reference will be made to the following detailed description of the invention which is to be read in association with the following drawings, wherein:

FIG. 1 is a perspective view of a percutaneous system embodying the present invention;

FIG. 2 is an enlarged partial side elevation in section showing the valve and diaphragm section of the invention implanted within a blood vessel with the diaphragm in a deflated condition;

FIG. 3 is a side elevation similar to FIG. 2 showing the diaphragm in an inflated condition;

FIG. 4 is a further enlarged view showing one end of the stent that supports the valves and diaphragm sections of the invention;

FIG. 5 is an enlarged partial view in section showing a weld joint for connecting two wire sections of the stent; and

FIG. 6 is an enlarged partial view showing the thickness of a vein which supports a biological valve being reduced in size to reduce the bulk of the implantation package.

### DETAILED DESCRIPTION OF THE INVENTION

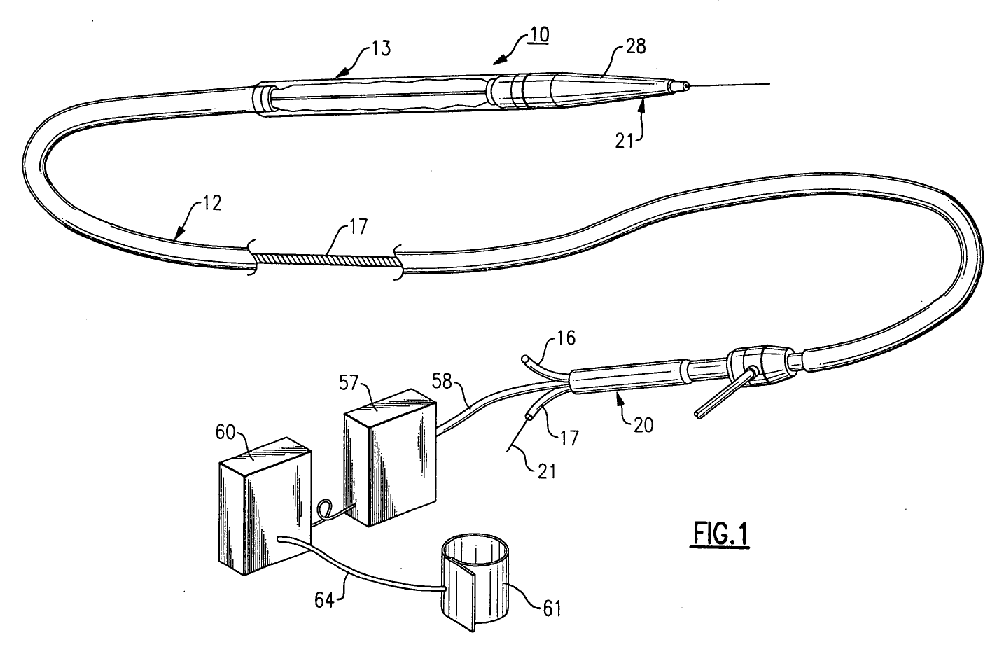

Referring now to the drawings, there is illustrated in FIG. 1 a system generally referenced 10 for the percutaneous implantation of a device for assisting the heart of a patient awaiting a heart transplant in circulating blood throughout the patient's body. The system includes an elongated catheter 12 that contains a balloon 13 at its distal end. The balloon is in fluid flow communication with a source of inflation through a lumen 16 that passes internally through the body of the catheter in a manner that is well known in the art. Preferably, the balloon is inflated using a radio opaque fluid so that its position within the patient's body can be more easily ascertained using well known tracking methods. Although a single balloon is employed in the practice of the present invention, it should be obvious to one skilled in the art that two or more superimposed balloons may be employed without departing from the teachings of the invention.

The catheter also includes a centrally positioned lumen 17 that passes through the entire length of the catheter from its proximal end 20 to its distal end. The central lumen is fabricated of a metal which is preferably a flexible thin-walled stainless steel tubing.

Maneuvering a balloon catheter through a blood vessel into an implantation site oftentimes becomes difficult particularly where the balloon package is bulky and the blood vessel has a number of turns and bends along the intended path of travel. Conventional catheters and the lumen running through the catheter body are fabricated from materials that tend to twist and bend when the catheter is placed under a bending stress or is twisted. Twisting and bending adversely effects the ability of the attending physician to quickly and accurately place the balloon package within the intended target region. The stainless steel tubing that runs down the center of the insertion tube is designed to provide the catheter with a 1-1 torque ratio. This means that any slight torque that is applied to the proximal end of the catheter will produce the same amount of torque at the distal end of the catheter. Although the stainless steel central lumen is thin enough so that it will have sufficient flexibility to transcend bends and turns in an implanted blood vessel, the tubing will also provide some amount of beam strength to the catheter such that the attending physician can more easily push the catheter along its intended path of travel. This along with the contoured catheter nosepiece 28 enhances the maneuverability of the catheter. A guide wire 29 passes through the central lumen and is used in a conventional manner to help guide the balloon package into the target region.

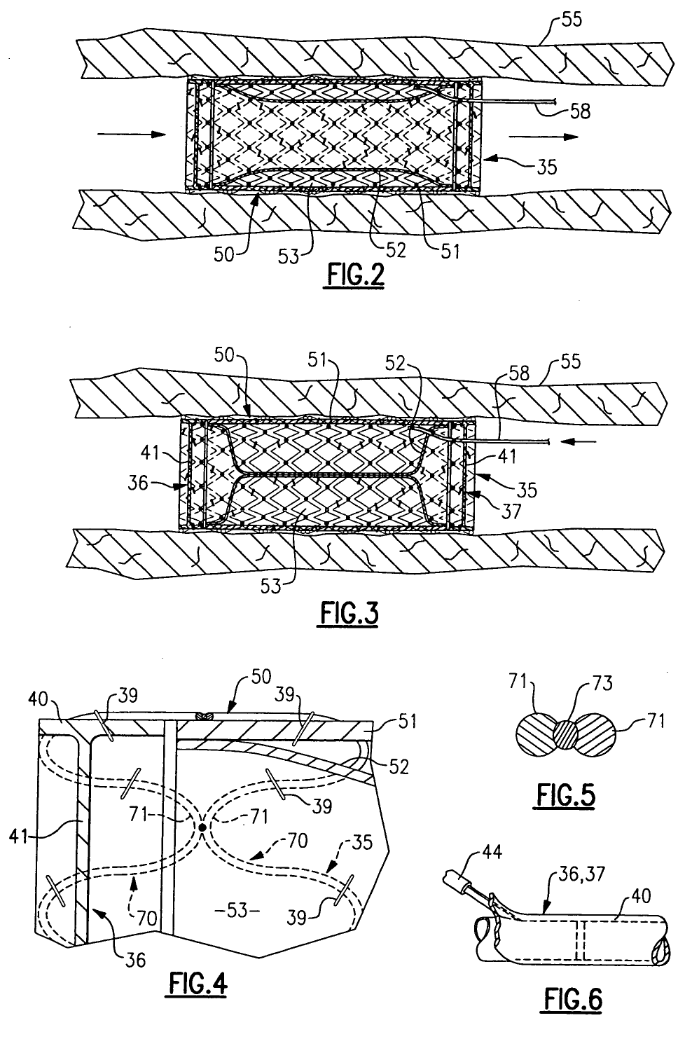

With further reference to FIGS. 2-4, a stent 35 is mounted over the balloon section of the catheter in a collapsed condition prior to insertion. Biological venous tricuspid valves 36 and 37 are secured to each end of the stent by suitable sutures 39-39. Each valve section includes a section of the vein 40 and a circular tricuspid valve 41 that is situated inside the vein section. Because of its makeup and geometry, each valve can be collapsed along with the stent to create a compact package capable of being percutaneously implanted within a blood vessel. As best illustrated in FIG. 6, the vein section 40 of each valve section is trimmed to a desired length and the wall of the vein is shaved as illustrated to reduce the thickness of the vein between fifty percent and ninety percent of its original thickness. This reduces considerably the size of each valve section when in a collapsed condition thereby reducing the overall size of the balloon package for greater ease of implantation. It has been found that the biological venous valves of certain animals are formed by layers of tissue. The layers, in turn, can be easily peeled back away from underlying layers by simply inserting a sharp instrument 44 between the layers in the manner illustrated in FIG. 6. The wall of each vein is shaved or otherwise cut back so that it approximates the inside diameter of the stent when the stent is expanded to a fully open position. The vein of each valve section is sutured to the stent when the stent is in a fully expanded condition as illustrated in FIGS. 2 and 3. The valves are mounted within the stent so that both valves will open in response to a flow of blood through the stent in one direction and close in response to a flow of blood in the opposite direction, the reason for which will become clear from the description below.

A cylindrical inflatable diaphragm 50 is also mounted inside the stent between the two venous valve sections again using sutures 39. The diaphragm includes an outer cylindrical membrane 51 and an inner cylindrical membrane 52 that are sealed together about the two opposing ends to create a chamber 53 therebetween. The inner membrane is fabricated of a resilient material such as Teflon, nylon, or EPTFe while the outer membrane is fabricated of a less resilient material such as polyurethane. The diaphragm like the venous valves can be collapsed with the stent over the balloon section of the catheter to establish a relatively small insertion package that will expand when the stent is expanded by the balloon into contact with a blood vessel 55, which can be either an artery leading to the heart or a vein leading away from the heart.

The diaphragm chamber is placed in fluid flow communication with a compact portable pump 57 by means of a flow line 58. The pump is arranged to deliver a fluid such as air or oxygen into the diaphragm chamber as illustrated in FIG. 2 to inflate the diaphragm as shown in FIG. 3 and then withdraw the fluid from the chamber to once again collapse the diaphragm. The diaphragm pump is connected to a controller 60 that contains a microprocessor for regulating the cycling of the pump in response to the rhythm of the patient's heart. The heart action is monitored by blood pressure cuff 61 or any other similar device capable of sensing the diastolic and systolic periods and providing the data to the controller via data line 64.

As noted above, the stent can be mounted either within an artery leading to the heart or a vein leading away from the heart to aid the flow of blood being carried to or away from the heart. The valves contained within the stent are arranged to permit the blood moving through the stent to flow in the normal circulatory direction while preventing blood from flowing in the opposite direction. Accordingly, the pump can be cycled to inflate the diaphragm during periods of the heart cycle which will most effectively assist the heart in circulating blood.

Preferably, the inner membrane of the diaphragm is fabricated of a Teflon material having a permeability that will permit a certain amount of the inflation fluid to pass through the membrane into the blood flow at inflation pressures. The inflation fluid is preferably oxygen, which can be absorbed directly into the bloodstream and carried to various parts of the body that need a supply of oxygen.

The present stent includes a series of fine wire ribbon sections 70. The sections are sinusoidal in shape and are co-joined to create a cylindrical shaped member when the stent is expanded. The wire strands making up each section is fabricated from a fully annealed malleable metal alloy containing about ninety percent platinum and ten percent iridium and having a tensile strength of between about 150,000 psi and 175,000 psi. Although a platinum iridium wire is preferred, other alloys having similar properties may be employed, such as some gold nickel alloys, without departing from the present invention. Each sinusoidal shaped ribbon section is wound into a cylinder and the cylinders are placed in axial alignment with the apex 71 of each bend in one cylinder being located adjacent that of a neighboring section in close proximity therewith. The bends of adjacent neighbors are then co-joined by welds 73 as illustrated in FIG. 5. The welds are contained inside the inner and outer boundaries described by the wire sections so that the welds do not protrude into regions where they might cause damage to the biological valves or the tissue of the containing blood vessel.

A stent that is constructed and configured as described herein will exhibit good flexibility, dimensional stability, a low profile when collapsed, and high immunity to fatigue and corrosion. In addition, the working range of the stent between a fully collapsed and a fully expanded condition can be easily varied by varying the number of bend sections in each circular ribbon section. As should be evident, the stent can be tailored for different applications to provide for the most effective implantation of diaphragm and valve combinations. The welds are further formed so that weld strength is less than that of the stent wire. In the event the stent for some reason becomes overly stressed, one or more welds in the stressed region will fracture before the wire can shatter, thus holding the potential of causing serious damage to a minimum.

While this invention has been particularly shown and described with reference to the preferred embodiment in the drawings, it will be understood by one skilled in the art that various changes in its details may be effected therein without departing from the teachings of the invention.

What is claimed is:

1. A percutaneous implanted apparatus for temporarily sustaining a patient awaiting a heart transplant that includes:
a radially expandable stent having a first collapsible valve secured to one end of said stent and a second collapsible valve secured to the other end of said stent, said valves being configured so that the valves open to pass a flow in one direction and close to block the flow in the opposite direction;
means for percutaneously implanting the stent into a blood vessel leading to or away from the heart and expanding the stent against the inner wall of the blood vessel;
a radially expandable diaphragm having a first cylindrical outer membrane secured to the stent between said valves and a second cylindrical inner membrane, said membranes being joined together at each end of said diaphragm to establish an inflatable chamber between the two membranes; and
means for inflating and deflating said diaphragm to assist the flow of blood being conducted through said blood vessel.

2. The apparatus of claim 1, wherein said means for implanting said stent is a balloon catheter.

3. The apparatus of claim 1, wherein said valves are biological venous valves, each of which is contained within a vein that is sutured to said stent.

4. The apparatus of claim 3, wherein the wall thickness of each vein is reduced to between 50 percent and 90 percent of its original thickness.

5. The apparatus of claim 1, wherein said means for inflating and deflating said diaphragm is a pump.

6. The apparatus of claim 5 that further includes control means for regulating said pump in response to the diastolic and systolic rhythm of the patient.

7. The apparatus of claim 1, wherein said inner membrane is fabricated of a material that is permeable to the inflation fluid at the pump pressure.

8. The apparatus of claim 7, wherein said outer membrane is fabricated of a polyurethane material and the inner membrane is fabricated of a material selected from the group consisting of Teflon, nylon, and EPTFe.

9. The apparatus of claim 1, wherein said outer membrane is joined to the inner membrane by a thermal bond.

10. The apparatus of claim 7, wherein said inflation fluid is oxygen.

11. A method of temporarily sustaining a patient awaiting a heart transplant that includes the steps of:
mounting a biological valve upon either end of a collapsible stent so that the valves operate to allow a fluid to flow in one direction through said stent and block the flow of said fluid in the opposite direction;
securing a radially expandable diaphragm to the inside of said stent between said valves;
connecting the diaphragm to a source for inflating and deflating said diaphragm;
percutaneously implanting the stent in a collapsed condition within a blood vessel leading to or away from the heart and expanding the stent into contact with the inner wall of the blood vessel; and
inflating and deflating said diaphragm in relation to the patient's diastolic and systolic rhythm.

12. The method of claim 11 that includes the further step of mounting the collapsed stent upon the balloon section of a balloon catheter for implanting the stent within said blood vessel.

13. The method of clam 11, wherein said biological valves are venous tricuspid valves and arranging said valves pass blood flowing in one direction and prevent the blood from flowing in the opposite direction.

14. The method of claim 11 that includes the further step of fabricating section of said diaphragm of a material that is permeable to oxygen at the inflation pressure.

15. The method of claim 14 that includes the further step of pumping oxygen into said diaphragm to inflate said diaphragm.

16. The method of claim 15 that includes the further steps of monitoring the heart's circulator rhythm and regulating the inflation and deflation of said diaphragm in response to said rhythm.
